(19) United States
(12) Patent Application Publication
(10) Pub. No.: US 2026/0152773 A1
(43) Pub. Date: Jun. 4, 2026

(54) METHOD FOR BIOLOGICAL PRETREATMENT OF HEMICELLULOSE-LIGNIN AND METHOD FOR ENZYMATIC SACCHARIFICATION COMPRISING THE SAME

(71) Applicant: KOREA INSTITUTE OF SCIENCE AND TECHNOLOGY, Seoul (KR)

(72) Inventors: Gyeongtaek GONG, Seoul (KR); Chang Soo KIM, Seoul (KR); Jong Hyuk PARK, Seoul (KR); Ja Kyong KO, Seoul (KR); Jung Ho AHN, Seoul (KR); Youngsoon UM, Seoul (KR)

(21) Appl. No.: 19/094,184

(22) Filed: Mar. 28, 2025

(30) Foreign Application Priority Data
Dec. 3, 2024 (KR) 10-2024-0177245

(51) Int. Cl. C12P 19/02

(52) U.S. Cl. CPC C12P 19/02; C12P 2201/00

(57) ABSTRACT

The present disclosure provides a composition for biological pretreatment of hemicellulose and lignin, comprising a red oleaginous yeast strain. Further, the present disclosure discloses a method for biological pretreatment of hemicellulose and lignin using a red oleaginous yeast strain, and a method for enzymatic saccharification comprising the same. According to the present disclosure, the efficiency of producing sugars from lignocellulosic biomass can be increased by biologically pretreating hemicellulose and lignin without sugar loss.

### CROSS REFERENCE TO RELATED APPLICATION

The present application claims priority to Korean Patent Application No. 10-2024-0177245, filed Dec. 3, 2024, the entire contents of which are hereby incorporated by this reference.

### BACKGROUND OF THE INVENTION

### Field of the Invention

The present specification describes a method for biological pretreatment of hemicellulose-lignin and a method for enzymatic saccharification comprising the same.

### Description about National Support Research and Development

This research was conducted through the following national projects.
Project Unique No.: 2710034017, Project No.: 2E33200, Ministry name: Ministry of Science and ICT, Project management (professional) organization name: National Research Foundation of Korea, Research Business Name: Korea Institute of Science and Technology Research Operating Expenses Support (Major Project Expenses), Research Project Name: Development of Core Technology of Electro Super Cellulose Composite Material, Name of project performing organization: Korea Institute of Science and Technology
Project Unique No.: 1711201840, Project No. CAP20024-300, Ministry name: Ministry of Science and ICT, Project management (professional) organization name: National Research Council of Science & Technology, Research Business Name: National Research Council of Science & Technology Research Council Research Operating Expenses Support (Major Project Expenses), Research Project Name: Development of a convergence solution for environmental contamination issues of secondary microplastics, Name of project performing organization: Korea Institute of Science and Technology
Project Unique No.: 2710013695, Project No.: 2020M1A2A2080847, Ministry name: Ministry of Science and ICT, Project management (professional) organization name: National Research Foundation of Korea, Research Business Name: Development of Technologies to Respond to Climate Change, Research Project Name: Development of medium-chain/long-chain fatty acid producing strains and bioprocesses for conversion of unused biomass, Name of project performing organization: Korea Institute of Science and Technology

### Description of the Related Art

Lignocellulosic biomass is the most abundant recyclable resource on earth and is composed of cellulose, hemicellulose and lignin. Such lignocellulosic biomass is not easily converted into sugar through a common saccharification process because the cellulose is tightly linked to the hemicellulose and lignin by various chemical bonds, so that a pretreatment process for the hemicellulose and lignin is required to increase the saccharification efficiency. As a method for pretreating hemicellulose and lignin, methods such as chemical pretreatment, biological pretreatment, fine pulverization, enzymatic treatment, hydrothermal treatment, acid treatment, or alkali treatment are known.

Although biological pretreatment has the advantages of being environmentally friendly, low cost, and not discharging toxic materials, and the like, it requires a long pretreatment period, there is a possibility of sugar consumption by pretreatment microorganisms, and it is necessary to optimize the pretreatment conditions and remove impurities for enzymatic saccharification treatment. In the case of technologies reported in the related art, since sugar loss occurs during the biological pretreatment process, the conversion rate into culturable sugars is low even though an excessive amount of enzymatic saccharification treatment is performed on biologically pretreated lignocellulosic biomass, so that there was a limitation that additional processes of washing with distilled water and high-temperature steam sterilization were required to improve the efficiency of enzymatic saccharification.

### SUMMARY OF THE INVENTION

A problem to be solved by the present disclosure is to provide a composition for biological pretreatment of hemicellulose-lignin that does not cause sugar loss and does not require an additional pretreatment process, and a biological pretreatment method using the same.

To solve the above problem, an embodiment of the present disclosure provides a composition for biological pretreatment of hemicellulose and lignin, comprising a red oleaginous yeast strain as an active ingredient.

Further, an embodiment of the present disclosure provides a method for enzymatic saccharification comprising: culturing a red oleaginous yeast strain in a medium containing hemicellulose and lignin to biologically pretreat the hemicellulose and lignin; and an enzymatic saccharification step comprising adding an enzyme to the biological pretreatment product of the hemicellulose and lignin obtained in the above step and culturing the biological pretreatment product.

According to an embodiment of the present disclosure, the efficiency of producing sugars from lignocellulosic biomass can be increased by biologically pretreating hemicellulose and lignin included in lignocellulosic biomass without sugar loss using a red oleaginous yeast strain. According to an embodiment of the present disclosure, fermentable sugars can be produced through biological pretreatment even under minimal medium conditions without the need for a long biological pretreatment period. In addition, since no washing processes such as additional washing with distilled water and high-temperature steam sterilization process are required after the biological pretreatment, there is an advantage in that enzymatic saccharification can be performed without any additional treatment after the biological pretreatment.

### BRIEF DESCRIPTION OF THE DRAWINGS

FIG. 1 is a view schematically illustrating a process for preparing a hemicellulose-lignin fractionated product (QMF) from lignocellulosic biomass according to an example of the present disclosure.

FIG. 2 is a view schematically illustrating a method for enzymatic saccharification according to an example of the present disclosure, the method comprising: biologically pretreating a hemicellulose-lignin fractionated product (QMF) using a red oleaginous yeast (biological treatment); and enzymatically saccharifying the same (enzymatic saccharification).

FIG. 3 is a view illustrating a comparison of sugar concentrations before and after enzymatic saccharification (CTec2 (−)/CTec2 (+)) using the CTec2 enzyme in a hemicellulose-lignin fractionated product (QMF Original) and a medium comprising the hemicellulose-lignin fractionated product (QMF 10D), as an example of the present disclosure.

FIG. 4A is a view illustrating the cell density after biological pretreatment of a hemicellulose-lignin fractionated product-containing medium (QMF 10D) using Rhodosporidium toruloides 7833, as an example of the present disclosure, compared to the control (Control).

FIG. 4B is a view illustrating a comparison of the concentrations of sugars included in a hemicellulose-lignin fractionated product-containing medium (QMF 10D) after biological pretreatment (after 168 hours) of the medium using Rhodosporidium toruloides 7833, as an example of the present disclosure.

FIG. 5A is a view illustrating the cell density after biological pretreatment of a hemicellulose-lignin fractionated product-containing medium (QMF 10D) using Rhodotorula glutinis 7989, as an example of the present disclosure, compared to the control (Control).

FIG. 5B is a view illustrating a comparison of the concentrations of sugars included in a hemicellulose-lignin fractionated product-containing medium (QMF 10D) after biological pretreatment (after 168 hours) of the medium using Rhodotorula glutinis 7989, as an example of the present disclosure.

FIG. 6A is a view illustrating the cell density after biological pretreatment of a hemicellulose-lignin fractionated product-containing medium (QMF 10D) using Rhodotorula glutinis var. dairenensis 17085, as an example of the present disclosure, compared to the control (Control).

FIG. 6B is a view illustrating a comparison of the concentrations of sugars included in a hemicellulose-lignin fractionated product-containing medium (QMF 10D) after biological pretreatment (after 168 hours) of the medium using Rhodotorula glutinis var. dairenensis 17085, as an example of the present disclosure.

FIG. 7A is a view illustrating the cell density after biological pretreatment of a hemicellulose-lignin fractionated product-containing medium (QMF 10D) using Rhodotorula glutinis 27048, as an example of the present disclosure, compared to the control (Control).

FIG. 7B is a view illustrating a comparison of the concentrations of sugars included in a hemicellulose-lignin fractionated product-containing medium (QMF 10D) after biological pretreatment (after 168 hours) of the medium using Rhodotorula glutinis 27048, as an example of the present disclosure.

FIG. 8A is a view illustrating the cell density after biological pretreatment of a hemicellulose-lignin fractionated product-containing medium (QMF 10D) using Rhodosporidium toruloides 50536, as an example of the present disclosure, compared to the control (Control).

FIG. 8B is a view illustrating a comparison of the concentrations of sugars included in a hemicellulose-lignin fractionated product-containing medium (QMF 10D) after biological pretreatment (after 168 hours) of the medium using Rhodosporidium toruloides 50536, as an example of the present disclosure.

FIG. 9A is a view illustrating the cell density after biological pretreatment of a hemicellulose-lignin fractionated product-containing medium (QMF 10D) using Rhodosporidium toruloides 50537, as an example of the present disclosure, compared to the control (Control).

FIG. 9B is a view illustrating a comparison of the concentrations of sugars included in a hemicellulose-lignin fractionated product-containing medium (QMF 10D) after biological pretreatment (after 168 hours) of the medium using Rhodosporidium toruloides 50537, as an example of the present disclosure.

FIG. 10A is a view illustrating the cell density after biological pretreatment of a hemicellulose-lignin fractionated product-containing medium (QMF 10D) using Rhodosporidium toruloides 50674, as an example of the present disclosure, compared to the control (Control).

FIG. 10B is a view illustrating a comparison of the concentrations of sugars included in a hemicellulose-lignin fractionated product-containing medium (QMF 10D) after biological pretreatment (after 168 hours) of the medium using Rhodosporidium toruloides 50674, as an example of the present disclosure.

FIG. 11A is a view illustrating the cell density after biological pretreatment of a hemicellulose-lignin fractionated product-containing medium (QMF 10D) using Rhodosporidium toruloides 11367, as an example of the present disclosure, compared to the control (Control).

FIG. 11B is a view illustrating a comparison of the concentrations of sugars included in a hemicellulose-lignin fractionated product-containing medium (QMF 10D) after biological pretreatment (after 168 hours) of the medium using Rhodosporidium toruloides 11367, as an example of the present disclosure.

FIG. 12 is a view collectively illustrating the results of analyzing the concentrations of glucose, xylose, arabinose, acetic acid, and ethanol contained in products obtained by biologically pretreating each hemicellulose and lignin fractionated product using the eight types of red oleaginous yeasts used as an example of the present disclosure after enzymatic saccharification with CTec2.

FIG. 13A is a view illustrating the cell density after biological pretreatment of a hemicellulose-lignin fractionated product-containing medium (QMF 10D) using Yarrowia lipolytica W29, as a comparative example of the present disclosure, compared to the control (Control).

FIG. 13B is a view illustrating a comparison of the concentrations of sugars included in a hemicellulose-lignin fractionated product-containing medium (QMF 10D) after biological pretreatment (after 168 hours) of the medium using Yarrowia lipolytica W29, as a comparative example of the present disclosure.

FIG. 14 is a view collectively illustrating the results of analyzing the concentrations of glucose, xylose, arabinose, acetic acid, and ethanol contained in products obtained by biologically pretreating each hemicellulose and lignin fractionated product using a red oleaginous yeast (27048) used as an example of the present disclosure and a comparative example (W29) after enzymatic saccharification with CTec2.

### DETAILED DESCRIPTION OF THE INVENTION

The examples of the present disclosure disclosed in the present specification are exemplified for the purpose of describing the examples of the present disclosure only, and the examples of the present disclosure may be carried out in various forms and should not be construed to be limited to the examples described herein. Since the present disclosure may have various changes and different forms, it should be understood that the examples are not intended to limit the present disclosure to specific disclosure forms and they include all the changes, equivalents and replacements included in the spirit and technical scope of the present disclosure. Singular expressions include plural expressions unless the context clearly indicates otherwise. In the present application, the term “comprise”, “include”, “contain” or “have” is intended to designate the presence of characteristics, materials, numbers, steps, and constituent elements described in the specification or a combination thereof, and does not exclude in advance a possibility of the presence or addition of one or more other characteristics or materials, numbers, steps, actions, and constituent elements, or a combination thereof.

In the present disclosure, the term “biological pretreatment of hemicellulose and lignin” refers to a microbial treatment process carried out in order to increase saccharification efficiency prior to enzymatic saccharification of lignocellulosic biomass containing hemicellulose and lignin in a method for enzymatic saccharification of the lignocellulosic biomass containing hemicellulose and lignin. Through the biological pretreatment, the bond structure of lignin-hemicellulose, and the like may be changed.

In the present disclosure, the term “saccharification” refers to the conversion of high molecular weight carbohydrates to lower molecular weight sugars by hydrolysis with the reaction of enzymes or acids.

An embodiment of the present disclosure may provide a composition for biological pretreatment of hemicellulose and lignin, comprising a red oleaginous yeast strain as an active ingredient.

In the present disclosure, the term “biological pretreatment of hemicellulose and lignin” refers to a microbial treatment process carried out in order to increase saccharification efficiency prior to enzymatic saccharification of lignocellulosic biomass containing hemicellulose and lignin in the enzymatic saccharification of the lignocellulosic biomass containing hemicellulose and lignin. According to an embodiment of the present disclosure, the biological pretreatment may change the bond structure of lignin-hemicellulose, thereby producing sugar without sugar loss. In addition, when a pretreatment product of hemicellulose and lignin biologically pretreated according to an embodiment of the present disclosure are enzymatically saccharified, fermentable sugars may be produced using only one saccharification enzyme, which is efficient because no additional treatment after the biological pretreatment is required.

In an embodiment, the biological pretreatment target of the composition may be, but is not limited to, lignocellulosic biomass, as long as it contains hemicellulose and lignin. Specifically, the biological pretreatment target of the composition may be the hemicellulose and lignin fractionated product of lignocellulosic biomass from the viewpoint in order to increase the efficiency of enzymatic saccharification. In an embodiment, the hemicellulose and lignin fractionated product may be obtained by removing cellulose from lignocellulosic biomass.

In an embodiment, the red oleaginous yeast strain may comprise one or more selected from the group consisting of Rhodosporidium toruloides, Rhodotorula glutinis, and Rhodotorula glutinis var. dairenensis. More specifically, the Rhodosporidium toruloides may be, for example, a strain designated by the Microorganism Accession No. KCTC 7833, KCCM 50536, KCCM 50537, KCCM 50674, or KCCM 11367, but is not limited thereto. Specifically, the Rhodotorula glutinis may be, for example, a strain designated by the Microorganism Accession No. KCTC 7989 or KCTC 27048, but is not limited thereto. Specifically, the Rhodotorula glutinis var. dairenensis may be, for example, a strain designated by the Microorganism Accession No. KCTC 17085, but is not limited thereto.

In an embodiment, the composition may further comprise a medium required for culturing the red oleaginous yeast strain. In an embodiment, the medium may be a minimal medium containing a nitrogen source, and specific examples of the medium are as described below.

In an embodiment, the concentration of the red oleaginous yeast strain contained in the composition may be expressed as an optical density value at 600 nm, and the optical density value may be specifically 0.01 to 16. In an embodiment, the optical density value may be 0.01 or more, 0.05 or more, 0.1 or more, 0.5 or more, 1 or more, 2 or more, 3 or more, 4 or more, 5 or more, 6 or more, 7 or more, 8 or more, 9 or more, 10 or more, 11 or more, 12 or more, 13 or more, 14 or more, or 15 or more, and may be 16 or less, 15 or less, 14 or less, 13 or less, 12 or less, 11 or less, 10 or less, 9 or less, 8 or less, 7 or less, 6 or less, 5 or less, 4 or less, 3 or less, 2 or less, 1 or less, 0.5 or less, 0.1 or less, or 0.05 or less. The concentration is the initial concentration of the yeast strain, which increases during the culture.

Another embodiment of the present disclosure may provide a method for biological pretreatment of hemicellulose and lignin using the composition. Specifically, the method may comprise culturing the red oleaginous yeast strain in a medium containing hemicellulose and lignin, and the culturing method is as described below.

Furthermore, an embodiment of the present disclosure may provide a method for enzymatic saccharification comprising: culturing the above-described red oleaginous yeast strain in a medium containing hemicellulose and lignin to biologically pretreat the hemicellulose and lignin; and an enzymatic saccharification step comprising adding an enzyme to the biological pretreatment product of the hemicellulose and lignin obtained in the above step and culturing the biological pretreatment product.

In the present disclosure, the “culture” can be applied without limitation as long as it is a culture method known in the art. As an embodiment, the microorganism may be cultured by any one selected from the group consisting of shaking culture, stationary culture, batch culture, fed batch culture, and continuous culture. The shaking culture refers to a method in which a culture inoculated with a microorganism is cultured while being shaken, and the stationary culture refers to a method in which a liquid culture inoculated with a microorganism is cultured while being left to stand without being shaken. The batch culture refers to a culture method in which a culture is cultured while fixing the volume of the culture is fixed and adding no new culture from the outside, and the fed batch culture refers to a culture method in which a small amount of elements are first added and then the raw materials are added little by little, in contrast to the single culture (batch) in which all the raw materials are first put into a culture tank and cultured. The continuous culture refers to a culture method in which a fresh nutrient medium is continuously supplied and at the same time a culture containing cells and products is continuously removed.

As an embodiment, the culture may be performed in a typical medium containing at least one selected from the group consisting of a carbon source, a nitrogen source, amino acids, vitamins, and the like, by meeting the culture requirements of the microorganism while adjusting temperature, pH, and the like. For example, the medium be a minimal medium or a Luria Bertani (LB) medium. As a nitrogen source that may be used as an embodiment, it is possible to use an inorganic nitrogen source such as ammonia, ammonium sulfate, ammonium chloride, ammonium acetate, ammonium phosphate, ammonium carbonate, and ammonium nitrate; and an organic nitrogen source such as an amino acids such as glutamic acid, methionine, and glutamine, and peptone, NZ-amine, a meat extract, a yeast extract, a malt extract, a corn steep liquor, a casein hydrolysate, a fish or a decomposition product thereof, and a defatted soybean cake or a decomposition product thereof. These nitrogen sources may be used either alone or in combination. The medium may contain potassium phosphate monobasic, potassium phosphate dibasic, and the corresponding sodium-containing salt as a component. The component that may be used as an embodiment comprises potassium dihydrogen phosphate or dipotassium hydrogen phosphate or the corresponding sodium-containing salt. Furthermore, as an inorganic compound, it is possible to use sodium chloride, calcium chloride, iron chloride, magnesium sulfate, iron sulfate, manganese sulfate, calcium carbonate, and the like. Finally, in addition to the above materials, materials such as amino acids and vitamins may be used. As an example, suitable precursors may be used in the culture medium. The aforementioned raw materials may be added to the culture during the culture process in an appropriate manner, such as a batch method, a feed batch method, or a continuous method, but are not particularly limited thereto. The pH of the culture may be adjusted using an appropriate concentration of a basic compound such as sodium hydroxide, potassium hydroxide, and ammonia, or an acid compound such as phosphoric acid or sulfuric acid in appropriate amounts and manners.

In an embodiment, the red oleaginous yeast strain may be comprised in the medium at a concentration of 0.01 to 16 optical density values at 600 nm. In an embodiment, the optical density value may be 0.01 or more, 0.05 or more, 0.1 or more, 0.5 or more, 1 or more, 2 or more, 3 or more, 4 or more, 5 or more, 6 or more, 7 or more, 8 or more, 9 or more, 10 or more, 11 or more, 12 or more, 13 or more, 14 or more, or 15 or more, and may be 16 or less, 15 or less, 14 or less, 13 or less, 12 or less, 11 or less, 10 or less, 9 or less, 8 or less, 7 or less, 6 or less, 5 or less, 4 or less, 3 or less, 2 or less, 1 or less, 0.5 or less, 0.1 or less, or 0.05 or less. The concentration is the initial concentration of the yeast strain, which increases during the culture.

In an embodiment, the culture temperature during the culture in the biological pretreatment step may be 20° C. or higher, 21° C. or higher, 22° C. or higher, 23° C. or higher, 24° C. or higher, 25° C. or higher, 26° C. or higher, 27° C. or higher, 28° C. or higher, 29° C. or higher, 30° C. or higher, 31° C. or higher, 32° C. or higher, 33° C. or higher, 34° C. or higher, 35° C. or higher, 36° C. or higher, 37° C. or higher, 38° C. or higher, or 39° C. or higher, and may be 40° C. or lower, 39° C. or lower, 38° C. or lower, 37° C. or lower, 36° C. or lower, 35° C. or lower, 34° C. or lower, 33° C. or lower, 32° C. or lower, 31° C. or lower, 30° C. or lower, 29° C. or lower, 28° C. or lower, 27° C. or lower, 26° C. or lower, 25° C. or lower, 24° C. or lower, 23° C. or lower, 22° C. or lower, or 21° C. or lower. In an embodiment, the culture period during the culture may be 1 day or more, 2 days or more, 3 days or more, 4 days or more, 5 days or more, 6 days or more, 7 days or more, 8 days or more, 9 days or more, or 10 days or more, 15 days or more, 20 days or more, or 25 days or more, and may be 30 days or less, 25 days or less, 20 days or less, 15 days or less, 10 days or less, 9 days or less, 8 days or less, 7 days or less, 6 days or less, 5 days or less, 4 days or less, 3 days or less, or 2 days or less.

As an embodiment, in the biological pretreatment step, the hemicellulose and lignin may be contained in an amount of 1 to 50 vol % based on the total volume of the medium. Specifically, the hemicellulose and lignin may be contained in an amount of 1 vol % or more, 3 vol % or more, 5 vol % or more, 7 vol % or more, 9 vol % or more, 11 vol % or more, 13 vol % or more, 15 vol % or more, 17 vol % or more, 19 vol % or more, 20 vol % or more, 25 vol % or more, 30 vol % or more, 35 vol % or more, 40 vol % or more, or 45 vol % or more, and 50 vol % or less, 45 vol % or less, 40 vol % or less, 35 vol % or less, 30 vol % or less, 25 vol % or less, 20 vol % or less, 18 vol % or less, 16 vol % or less, 15 vol % or less, 13 vol % or less, 11 vol % or less, 9 vol % or less, 7 vol % or less, 5 vol % or less, or 3 vol % or less, based on the total volume of the medium.

In an embodiment, the method may further comprise removing cellulose from the lignocellulosic biomass to fractionate the hemicellulose and lignin prior to the step of biologically pretreating the hemicellulose and lignin. In an embodiment, the step of removing cellulose from the lignocellulosic biomass to fractionate hemicellulose and lignin may be performed by methods such as organic solvent extraction, chemical extraction, centrifugation, purification, and filtration, but is not limited thereto as long as the method can fractionate hemicellulose and lignin.

Specifically, the step of fractionating the hemicellulose and lignin may comprise reacting the lignocellulosic biomass with a mixture of water and an organic solvent, and then removing the cellulose. For example, the water and organic solvent mixture may be a mixed solution of water and ethanol. For example, the mixing ratio of the water and the organic solvent may be 1:1 to 10, more specifically 1:1. In one embodiment, the reaction may be carried out under the conditions of 185° C. to 195° C. and 10 to 30 bar. In an embodiment, the cellulose may be removed using a flow-through separator after the reaction is completed. Once the cellulose fraction has been removed, only the hemicellulose-lignin fraction remains in the solvent, so that a hemicellulose-lignin fractionated product may be obtained.

In an embodiment, the method may further comprise centrifuging the hemicellulose and lignin fractionated product to recover a supernatant from the viewpoint in order to increase the efficiency of enzymatic saccharification through biological pretreatment. In this case, the ratio of the supernatant may be 1 to 50 vol % based on the total volume of the entire fractionated product. Specifically, the ratio may be 1 vol % or more, 3 vol % or more, 5 vol % or more, 7 vol % or more, 9 vol % or more, 10 vol % or more, 15 vol % or more, 20 vol % or more, 25 vol % or more, 30 vol % or more, 35 vol % or more, 40 vol % or more, or 45 vol % or more, and 50 vol % or less, 45 vol % or less, 40 vol % or less, 35 vol % or less, 30 vol % or less, 25 vol % or less, 20 vol % or less, 15 vol % or less, 10 vol % or less, 9 vol % or less, 7 vol % or less, 5 vol % or less, or 3 vol % or less, based on the total volume of the entire fractionated product.

Further, in an embodiment, the method may further comprise, after the step of biologically pretreating the hemicellulose and lignin, separating and removing the red oleaginous yeast strain from a culture solution obtained in the biological pretreatment step to obtain a biological pretreatment product of hemicellulose and lignin.

In an embodiment, the biological pretreatment product of hemicellulose and lignin may consume all monosaccharides, acetic acid, and ethanol in the medium by the red oleaginous yeast strain.

In an embodiment, the separation step may be performed using methods well known in the art. For example, the separation method comprises a method such as centrifugation, sonication, organic solvent extraction, enzymatic extraction, mechanical extraction, and chemical extraction, but is not limited thereto.

In an embodiment, the enzyme used in the enzymatic saccharification step is not limited as long as it is a type capable of saccharifying the biological pretreatment product of hemicellulose and lignin. For example, the enzyme may be one of cellulase, hemicellulase, and xylanase.

In an embodiment, the culture temperature during the culture in the enzymatic saccharification step may be 40° C. or higher, 45° C. or higher, 46° C. or higher, 47° C. or higher, 48° C. or higher, 49° C. or higher, 50° C. or higher, 51° C. or higher, 52° C. or higher, 53° C. or higher, 54° C. or higher, 55° C. or higher, 57° C. or higher, or 59° C. or higher, and 60° C. or lower, 57° C. or lower, 55° C. or lower, 54° C. or lower, 53° C. or lower, 52° C. or lower, 51° C. or lower, 50° C. or lower, 49° C. or lower, 48° C. or lower, 47° C. or lower, 46° C. or lower, 45° C. or lower, 43° C. or lower, or 41° C. or lower. In an embodiment, the culture period may be 0.5 days (12 hours) or more, 1 day or more, 2 days or more, 3 days or more, 4 days or more, 5 days or more, 6 days or more, 7 days or more, 8 days or more, or 9 days or more, and 10 days or less, 9 days or less, 8 days or less, 7 days or less, 6 days or less, 5 days or less, 4 days or less, 3 days or less, 2 days or less, or 1 day or less.

The fermentable sugars obtained by the enzymatic saccharification method according to an embodiment may be used to produce high value-added compounds such as biodiesel and antioxidants (carotenoids).

Hereinafter, the configuration and effects of the present disclosure will be described more specifically with reference to examples and drawings. However, the following examples and drawings are provided for illustrative purposes only to aid in the understanding of the present disclosure, and the scope and spirit of the present disclosure are not limited thereby.

### Preparation Example 1

### Preparation of Hemicellulose and Lignin Fractionated Product

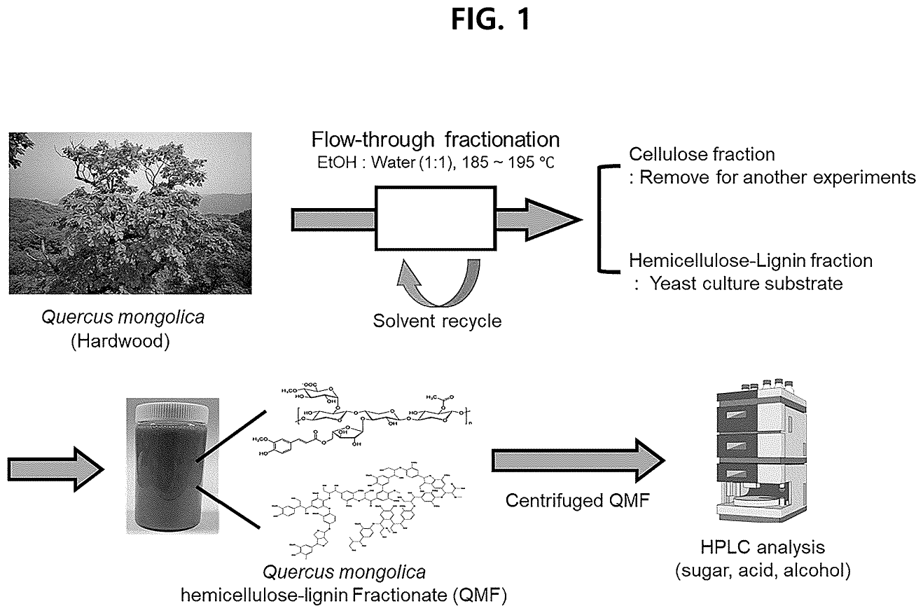

Hemicellulose and lignin fractionated products were prepared by the following method using Mongolian oak as the lignocellulosic biomass according to an embodiment of the present disclosure. FIG. 1 schematically illustrates the preparation method thereof.

First, Mongolian oak (scientific name: Quercus mongolica) as biomass was mixed with a mixed solution of water and ethanol at a volume ratio of 1:1. The biomass mixture was reacted under the conditions of 185° C. to 195° C. and 20 bar. After the reaction was completed, the cellulose fraction was removed using a flow-through separator, and only the hemicellulose and lignin fractions were allowed to remain in the ethanol mixed solution to obtain a Quercus mongolica hemicellulose-lignin fractionated product (QMF).

The Quercus mongolica hemicellulose-lignin fractionated product (QMF) from which the cellulose fraction was removed as described above was centrifuged for component analysis, and a supernatant was recovered through solid-liquid separation.

### Example 1

### Biological Pretreatment and Enzymatic Saccharification of Hemicellulose-Lignin Fractionated Product

As an embodiment, the Quercus mongolica hemicellulose-lignin fractionated product (QMF) prepared in the Preparation Example was centrifuged to recover the supernatant, which was then used for biological pretreatment using a red oleaginous yeast.

A yeast nitrogen source minimal medium was prepared as a medium for biological pretreatment using a red oleaginous yeast, and the prepared Quercus mongolica hemicellulose-lignin fractionated product was added to the yeast nitrogen source minimal medium at a ratio of 10% (vol/vol) to prepare a hemicellulose-lignin fractionated product-containing medium (QMF 10D). The yeast nitrogen source minimal medium used above comprises a yeast nitrogen source (YNB, Yeast nitrogen base without amino acid and ammonium sulfate, Sigma), an amino acid mixture (CSM, Complete Supplement Mixture, MP Bio), and a yeast extract (BD Difco).

As the red oleaginous yeast, a total of eight strains were used: Rhodosporidium toruloides KCTC 7833, KCCM 50536, KCCM 50537, KCCM 50674, and KCCM 11367, Rhodotorula glutinis KCTC 7989 and KCTC 27048, and Rhodotorula glutinis var. dairenensis KCTC 17085.

A biological pretreatment was performed by adding the yeast extract of each of the red oleaginous yeasts to the above-prepared hemicellulose-lignin fractionated product-containing medium (QMF10D) at a concentration of 1.5 g/L, and culturing the mixture under the conditions of 30° C. and 200 rpm for 7 days.

The culture solution completely biologically pretreated was centrifuged to separate and remove the red oleaginous yeast. To the culture solution from which the red oleaginous yeast had been removed, 0.281 mL of Cellic CTec2 (manufacturer: Novozymes) enzyme solution, a cellulase commercial enzyme, was added as a saccharification enzyme based on 10 mL of the culture solution, and the resulting mixture was cultured under the conditions of 50° C. and 200 rpm for 3 days to carry out an enzymatic saccharification treatment. The Cellic CTec2 is an enzyme mixture that is a mixture of cellulase, beta-glucosidase and hemicellulase.

After the enzymatic saccharification treatment was completed, the concentrations of glucose, xylose, arabinose, mannose, acetic acid, and ethanol contained in the enzymatic saccharification solution were analyzed using high-performance liquid chromatograph (HPLC) (manufacturer: YOUNGIN Chromass) and an Aminex 87H column (manufacturer: Bio-Rad).

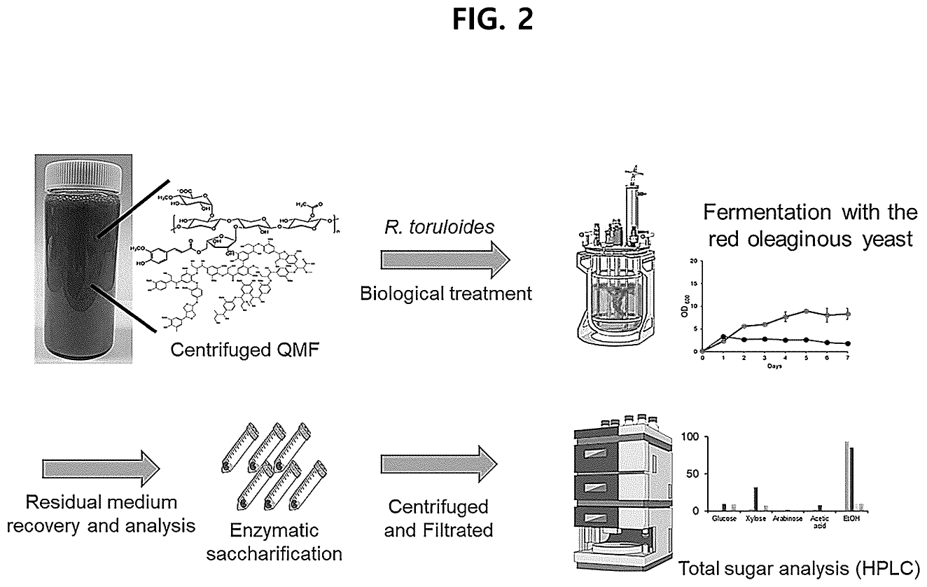

FIG. 2 is a view schematically illustrating the biological pretreatment and enzymatic saccharification steps described above.

### Test Example 1

Before confirming the effect of the biological pretreatment of the present disclosure, the composition of a hemicellulose-lignin fractionated product before and after enzymatic saccharification as a control was analyzed as follows.

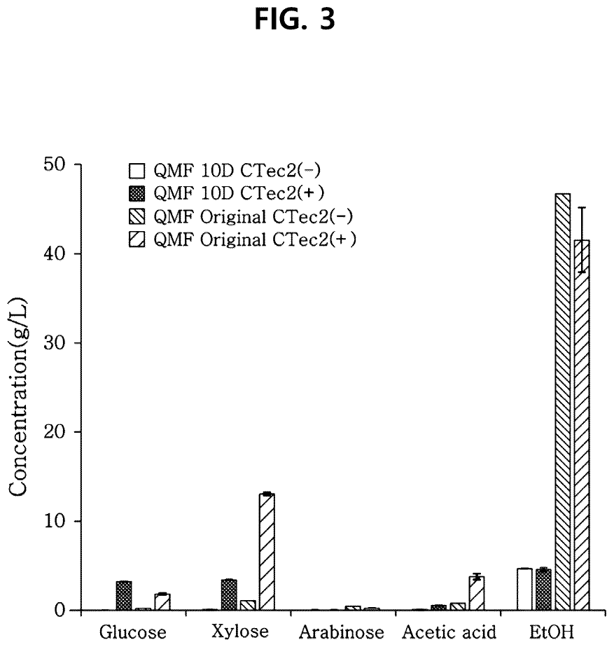

As samples to be analyzed, the hemicellulose-lignin fractionated product (QMF Original) prepared in Preparation Example 1 above and a hemicellulose-lignin fractionated product-containing medium (QMF10D) obtained by centrifuging the fractionated product to separate solids and liquids were prepared. Next, when each of the hemicellulose-lignin fractionated products (QMF Original) and the hemicellulose-lignin fractionated product-containing medium (QMF 10D) were enzymatically saccharified without biological pretreatment, the concentrations of components before and after enzymatic saccharification were analyzed using high-performance liquid chromatograph (HPLC)(manufacturer: YOUNGIN Chromass) and an Aminex 87H column (manufacturer: Bio-Rad), and the results are shown in FIG. 3. In this case, the minimum medium was the same as that used in Example 1, and the enzymatic saccharification was also carried out in the same manner as in Example 1.

As a result, the hemicellulose-lignin fractionated product that had not been subjected to enzymatic saccharification treatment (QMF Original CTec2 (−)) comprised glucose (0.19 g/L), xylose (1.07 g/L), arabinose (0.44 g/L), acetic acid (0.77 g/L), and ethanol (46.7 g/L), and after enzymatic saccharification treatment (QMF Original CTEC2 (+)), glucose, xylose, and acetic acid increased by 9.8-fold, 12-fold, and 4.9-fold, respectively, and arabinose and ethanol decreased by 0.17 g/L and 5.14 g/L, respectively.

Further, the hemicellulose-lignin fractionated product-containing medium that had not been subjected to enzymatic saccharification treatment (QMF 10D CTec2 (−)) comprised glucose (0.02 g/L), xylose (0.1 g/L), arabinose (0.03 g/L), acetic acid (0.1 g/L), and ethanol (4.7 g/L), and after enzymatic saccharification treatment (QMF 10D CTec2 (+)), glucose, xylose, and acetic acid increased by 162-fold, 34-fold, and 5.7-fold, respectively.

### Test Example 2

As an embodiment of the present disclosure, biological pretreatment and enzymatic saccharification using eight red oleaginous yeast strains were carried out in a hemicellulose-lignin fractionated product-containing medium (QMF10D) in the same manner as in Example 1 above, and the concentrations of glucose, xylose, arabinose, mannose, acetic acid, and ethanol contained in each saccharified product were analyzed.

In this case, as a control, a product obtained by adding each of the 8 yeast extracts to the same medium at a concentration of 1.5 g/L and culturing the yeast extract was used, except that the yeast extract did not comprise the Quercus mongolica hemicellulose-lignin fractionated product in Example 1.

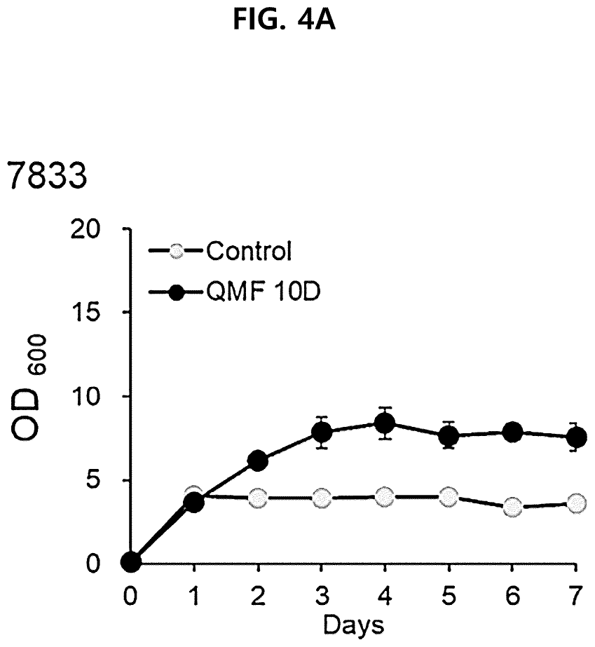

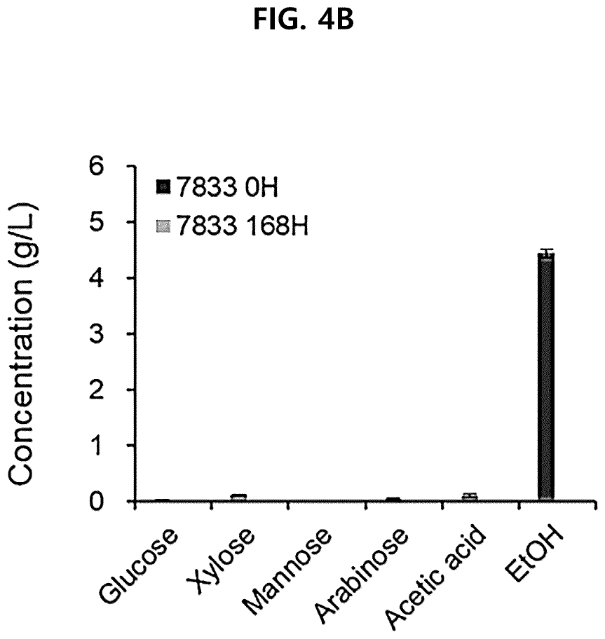

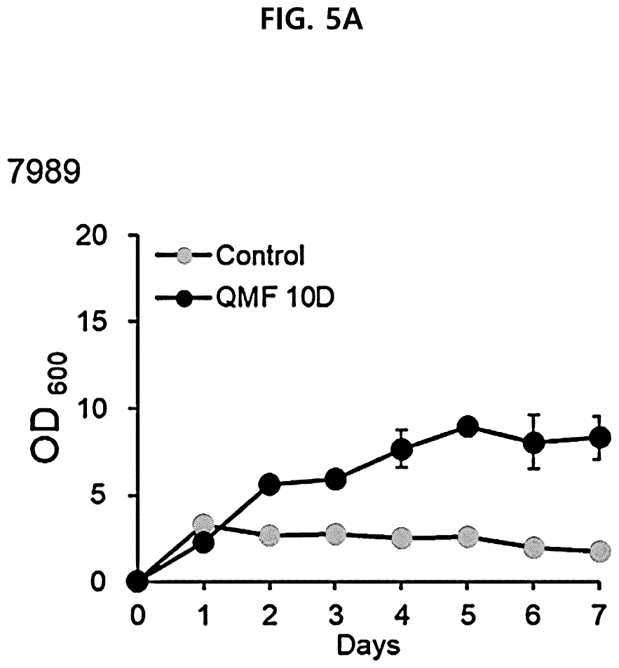

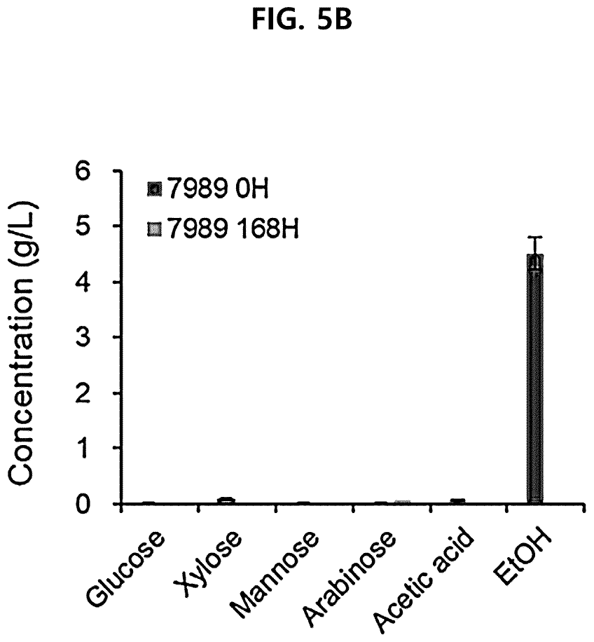

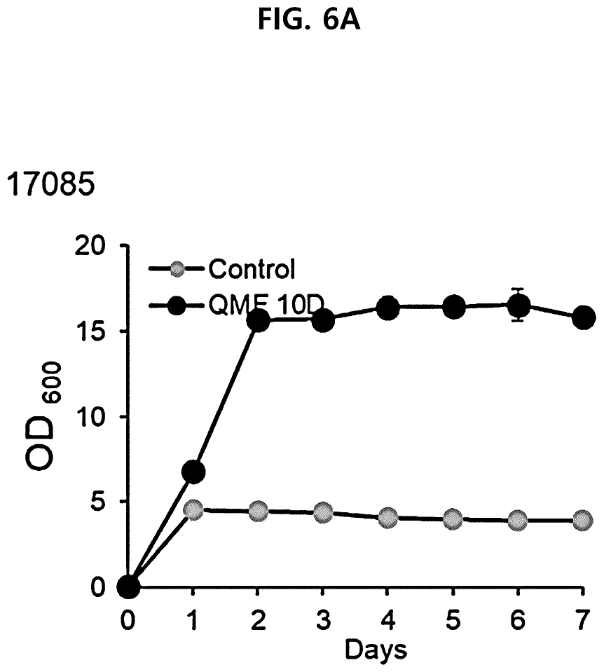

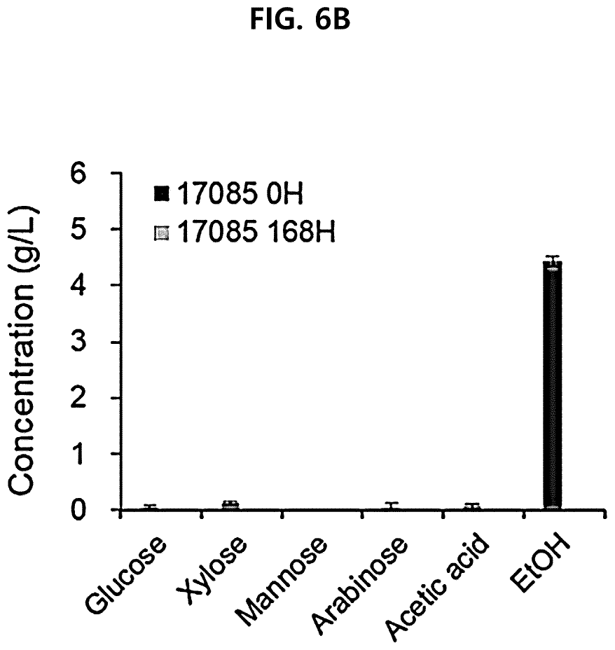

As a result, the red oleaginous yeast strains, Rhodosporidium toruloides KCTC 7833, Rhodotorula glutinis KCTC 7989, Rhodotorula glutinis var. dairenensis KCTC 17085, and Rhodotorula glutinis KCTC 27048, recorded cell densities 2.1-fold, 4.7-fold, 4.1-fold, and 5.7-fold higher than the control, respectively, in the culture medium without growth inhibition (FIGS. 4A to 7A). In addition, after 168 hours of culture (pretreatment), it was confirmed that monosaccharides, acetic acid, and ethanol present in each medium had been completely consumed and removed (FIGS. 4B to 7B).

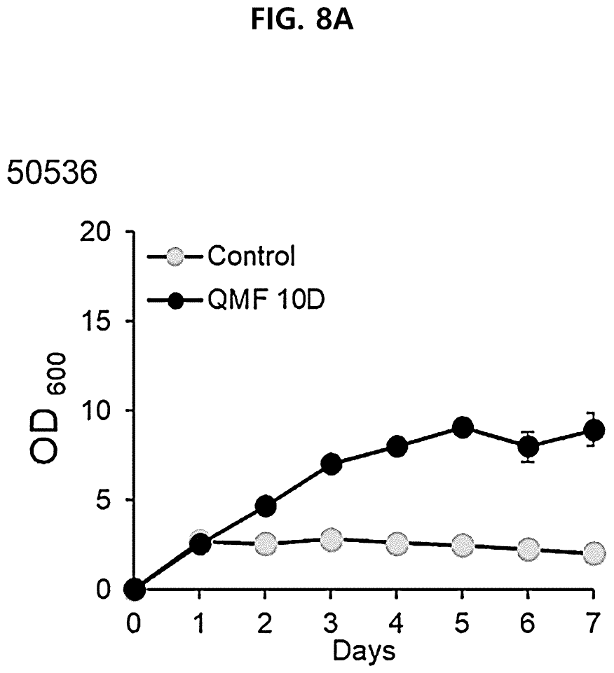

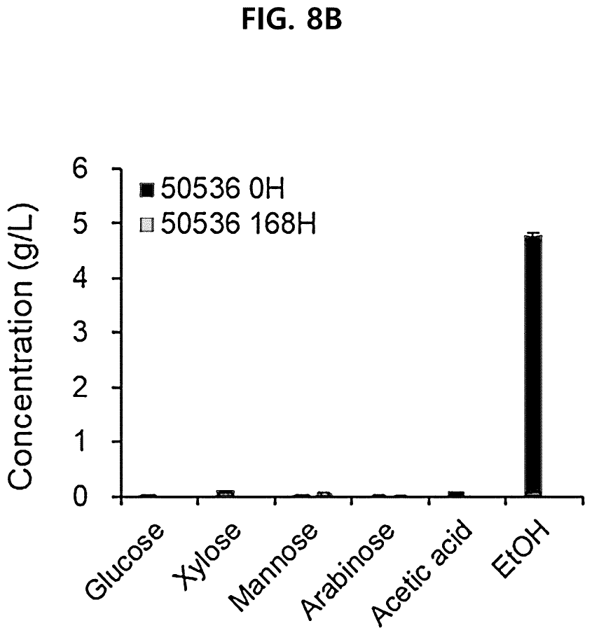

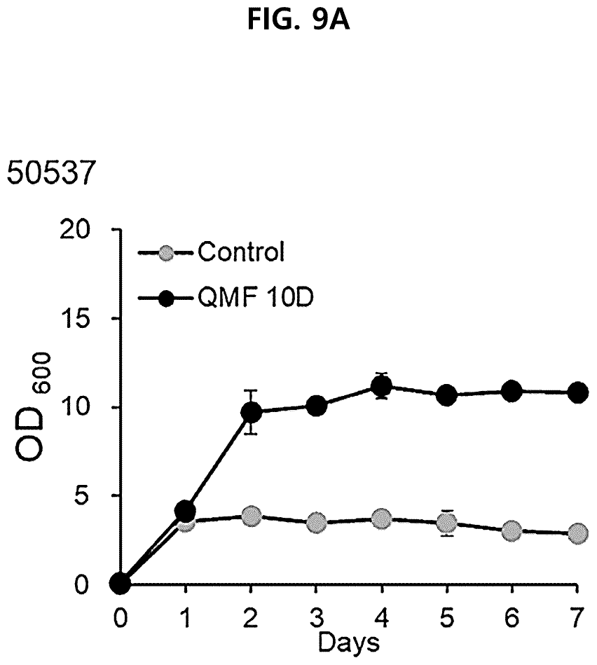

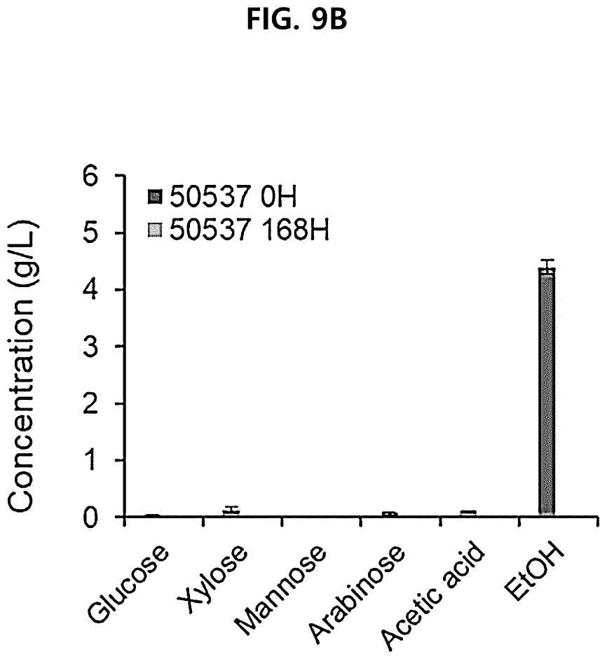

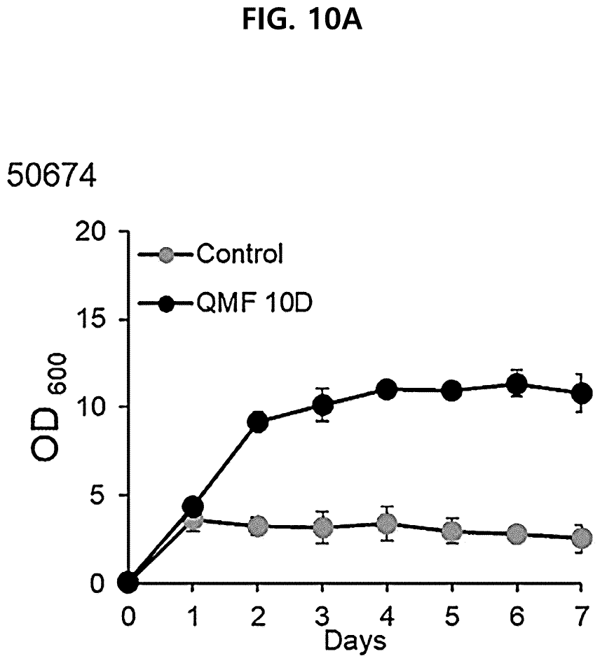

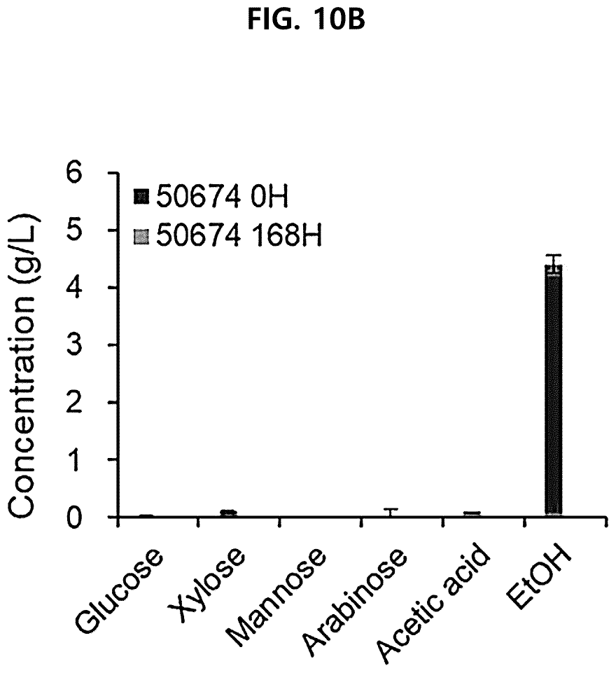

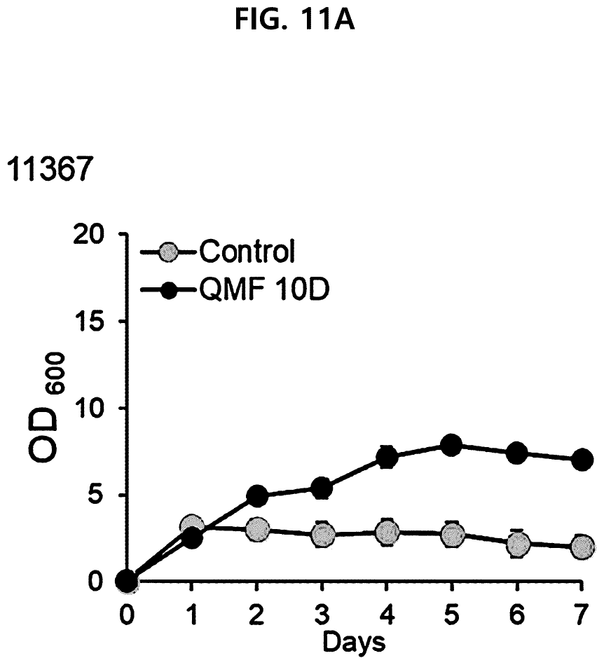

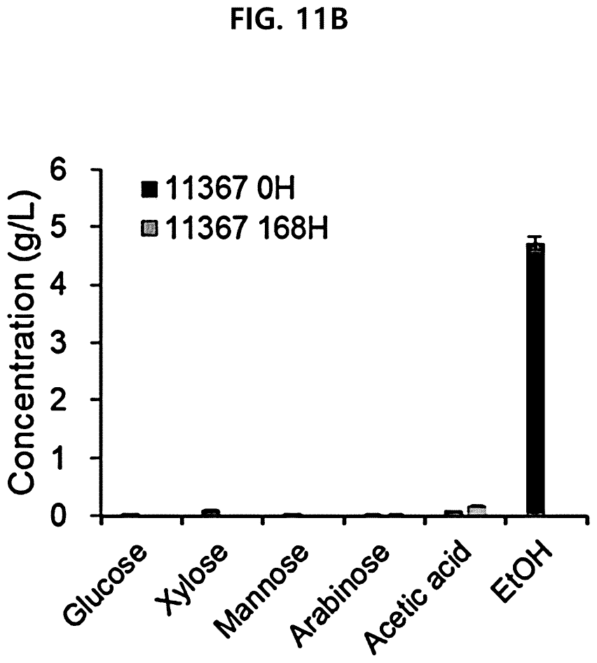

Furthermore, the red oleaginous yeast strains, Rhodosporidium toruloides KCCM 50536, KCCM 50537, KCCM 50674, and KCCM 11367, recorded cell densities 4.4-fold, 3.7-fold, 4.2-fold, and 3.5-fold higher than the control, respectively, in the culture medium without growth inhibition (FIGS. 8A to 11A). Further, after 168 hours of culture (pretreatment), it was confirmed that monosaccharides, acetic acid, and ethanol present in each medium had been completely consumed and removed (FIGS. 8B to 11B).

The above results mean that when red oleaginous yeast strains are used to carry out biological pretreatment, the efficiency of enzymatic saccharification of hemicellulose can be improved and increased without sugar loss.

### Test Example 3

As an embodiment of the present disclosure, enzymatic saccharification was performed using the biological pretreatment products prepared using each of the eight red oleaginous yeast strains obtained in Test Example 2 above, and the concentrations of glucose, xylose, arabinose, mannose, acetic acid, and ethanol contained in each enzymatic saccharification solution were analyzed. In this case, the enzymatic saccharification was carried out in the same manner as the method described in Example 1. As a control, a product obtained by performing enzymatic saccharification in the same manner as in Test Example 2 was prepared, except that the biological pretreatment was performed.

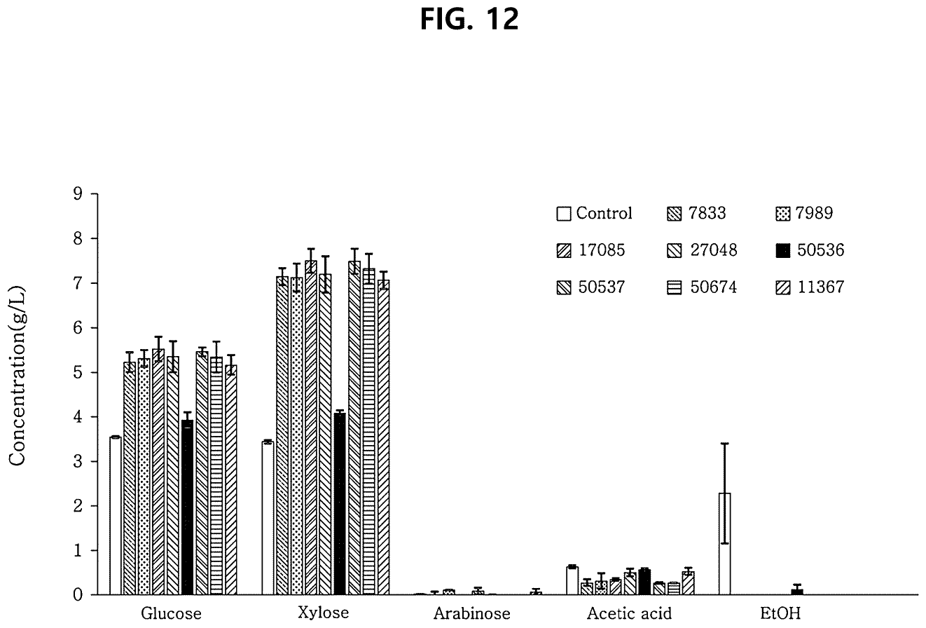

As a result, as shown in FIG. 12, in the case of the control in which enzymatic saccharification treatment was performed without biological pretreatment in the hemicellulose-lignin fractionated product culture medium, the glucose, xylose, arabinose, acetic acid, and ethanol were measured at 3.54 g/L, 3.44 g/L, 0.03 g/L, 0.64 g/L, and 2.28 g/L, respectively. In comparison, in the examples (7833, 7989, 17085, 27048, 50536, 50537, 50674, 11367) in which both biological pretreatment and enzymatic saccharification of hemicellulose-lignin fractionated products using red oleaginous yeasts were performed, the efficiency of producing monosaccharides was remarkably increased. Specifically, glucose increased by up to 1.6-fold and xylose increased by up to 2.2-fold depending on the strain, and almost no ethanol was comprised.

This means that when the method of the present disclosure is followed, excellent enzymatic saccharification effects can be obtained by performing enzymatic saccharification alone without any additional processes other than sugar loss or biological pretreatment.

### Test Example 4

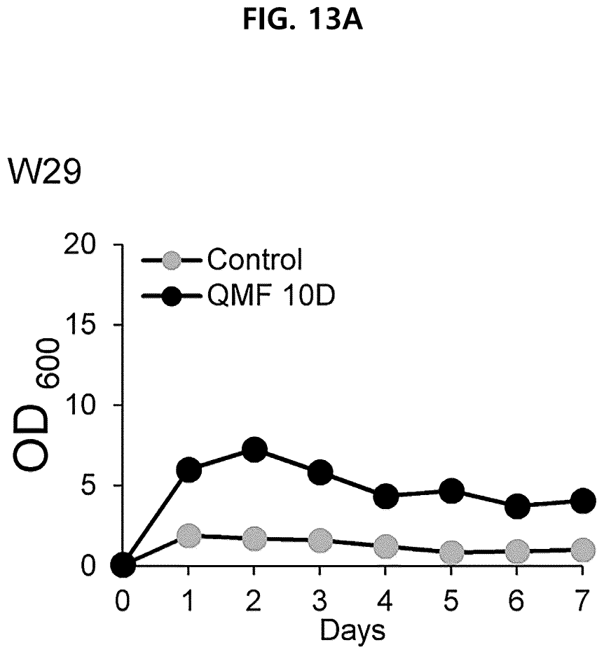

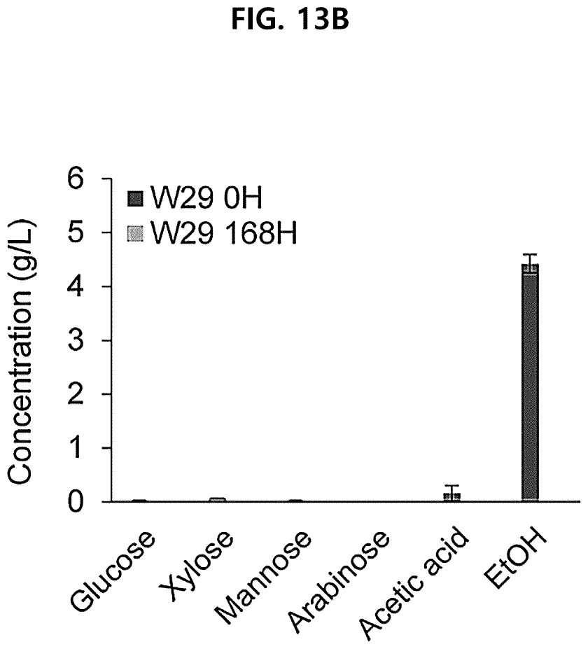

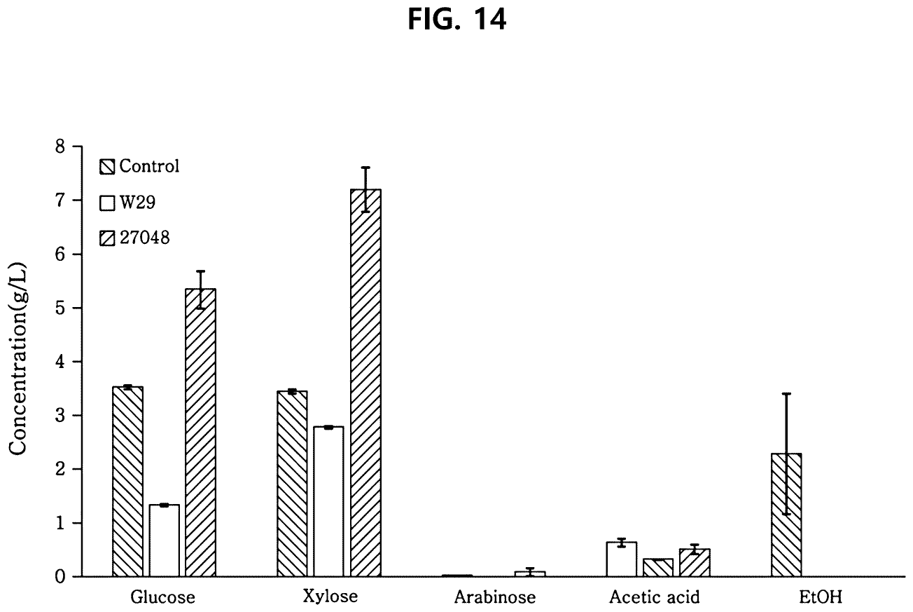

As a comparative example of the present disclosure, biological pretreatment was carried out in the same manner as in Test Example 2 above using Yarrowia lipolytica W29, which is known to be used in biological pretreatment and enzymatic saccharification of lignocellulosic biomass, analysis was performed, and the results are shown in FIGS. 13A and 13B. Moreover, biological pretreatment and enzymatic saccharification were performed in the same manner as in Test Example 3 above, and the results are shown in FIG. 14.

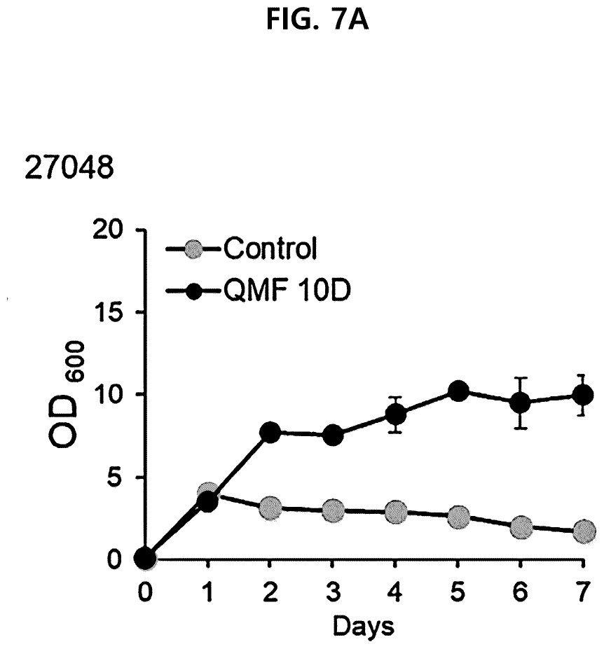

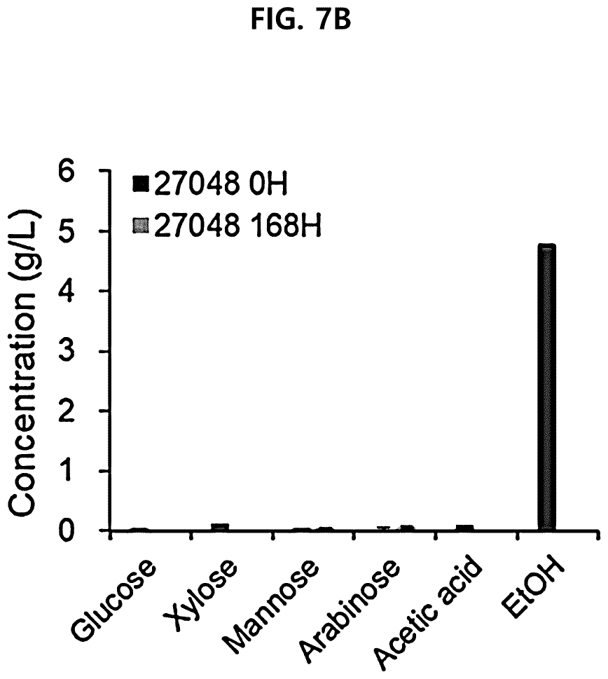

As an embodiment of the present disclosure, the results of biological pretreatment (FIGS. 7A and 7B) and the results of biological pretreatment and enzymatic saccharification (FIG. 12) of the red oleaginous yeast strain (Rhodotorula glutinis KCTC 27048) obtained in Test Examples 2 and 3 above were compared with the results of the Comparative Example.

As a result, the Yarrowia lipolytica W29 strain of the Comparative Example consumed all of the glucose, xylose, acetic acid, and ethanol in the culture medium (QMF 10D) during biological pretreatment, and had a cell density 4.01-fold higher than the control (Control), but the cell growth was shown to be remarkably lower than that of the red oleaginous yeast strain (27048) according to an embodiment of the present disclosure.

Furthermore, as shown in FIG. 14, when enzymatic saccharification was carried out after biological pretreatment using the Yarrowia lipolytica W29 strain of the Comparative Example, the concentrations of glucose, xylose, and acetic acid were 1.33 g/L, 2.78 g/L, and 0.32 g/L, respectively, confirming that the concentrations of glucose and xylose were 62% and 19% lower, respectively, than the control that was not biologically pretreated.

Conversely, when enzymatic saccharification was performed after biological pretreatment using the strain of the red oleaginous yeast strain (Rhodotorula glutinis KCTC 27048) according to an embodiment of the present disclosure, the amounts of glucose and xylose produced were increased by 4.01-fold and 2.59-fold, respectively, compared to the Yarrowia lipolytica W29 strain of the Comparative Example.

This means that when the red oleaginous yeast strain according to an embodiment of the present disclosure is used, it is possible to obtain an enzymatic saccharification efficiency that is remarkably improved compared to other strains previously reported for the same use. The present disclosure may provide the following embodiments as an example.

### First Embodiment

A composition for biological pretreatment of hemicellulose and lignin, comprising a red oleaginous yeast strain as an active ingredient.

### Second Embodiment

The composition of First Embodiment, wherein the red oleaginous yeast strain comprises one or more selected from the group consisting of Rhodosporidium toruloides, Rhodotorula glutinis, and Rhodotorula glutinis var. dairenensis.

### Third Embodiment

The composition of First or Second Embodiment, further comprising a minimal medium comprising a nitrogen source.

### Fourth Embodiment

The composition of any one of First to Third Embodiments, wherein the concentration of the red oleaginous yeast strain comprised in the composition has an optical density value of 0.01 to 16 at 600 nm.

### Fifth Embodiment

The composition of any one of First to Fourth Embodiments, wherein a target of the composition to be biologically pretreated is lignocellulosic biomass.

### Sixth Embodiment

The composition of any one of First to Fourth Embodiments, wherein a target of the composition to be biologically pretreated is a hemicellulose and lignin fractionated product of the lignocellulosic biomass.

### Seventh Embodiment

A method for enzymatic saccharification, the method comprising: culturing a red oleaginous yeast strain in a medium comprising hemicellulose and lignin to biologically pretreat the hemicellulose and lignin; and
an enzymatic saccharification step comprising adding an enzyme to a biological pretreatment product of the hemicellulose and lignin obtained in the step and culturing the biological pretreatment product.

### Eighth Embodiment

The method of Seventh Embodiment, further comprising,
after the step of biologically pretreating the hemicellulose and lignin, separating and removing a red oleaginous yeast strain from a culture solution obtained in the biological pretreatment step to obtain a biological pretreatment product of hemicellulose and lignin.

### Ninth Embodiment

The method of Seventh or Eighth Embodiment, wherein the medium in the biological pretreatment step is a minimal medium comprising a nitrogen source.

### Tenth Embodiment

The method of any one of Seventh to Ninth Embodiments, wherein in the biological pretreatment step, the hemicellulose and lignin are comprised in an amount of 1 to 50 vol % based on the total volume of the medium.

### Eleventh Embodiment

The method of Seventh to Tenth Embodiments, further comprising:
before the step of biologically pretreating the hemicellulose and lignin, removing cellulose from lignocellulosic biomass to fractionate hemicellulose and lignin.

### Twelfth Embodiment

The method of any one of Seventh to Eleventh Embodiments, wherein the red oleaginous yeast strain comprises one or more selected from the group consisting of Rhodosporidium toruloides, Rhodotorula glutinis, and Rhodotorula glutinis var. dairenensis.

### Thirteenth Embodiment

The method of any one of Seventh to Twelfth Embodiments, wherein the red oleaginous yeast strain is comprised at a concentration of an optical density value of 0.01 to 16 at 600 nm in the medium.

### Fourteenth Embodiment

The method of any one of Seventh to Thirteenth Embodiments, wherein for the biological pretreatment product of the hemicellulose and lignin, monosaccharides, acetic acid, and ethanol in the medium are completely consumed by the red oleaginous yeast strain.

### Fifteenth Embodiment

The method of any one of Seventh to Fourteenth Embodiments, wherein the culturing in the biological pretreatment step is performed at 20° C. to 40° C. for 1 to 30 days.

### Sixteenth Embodiment

The method of any one of Seventh to Fifteen Embodiments, wherein the culturing in the enzymatic saccharification step is performed at 40° C. to 60° C. for 0.5 to 10 days.

What is claimed is:

1. A method for enzymatic saccharification, the method comprising:
culturing a red oleaginous yeast strain in a medium comprising hemicellulose and lignin to biologically pretreat the hemicellulose and lignin; and
an enzymatic saccharification step comprising adding an enzyme to a biological pretreatment product of the hemicellulose and lignin obtained in the step and culturing the biological pretreatment product.

2. The method of claim 1, further comprising, after the step of biologically pretreating the hemicellulose and lignin, separating and removing the red oleaginous yeast strain from a culture solution obtained in the biological pretreatment step to obtain a biological pretreatment product of hemicellulose and lignin.

3. The method of claim 1, wherein the medium in the biological pretreatment step is a minimal medium comprising a nitrogen source.

4. The method of claim 1, wherein in the biological pretreatment step, the hemicellulose and lignin are comprised in an amount of 1 to 50 vol % based on a total volume of the medium.

5. The method of claim 1, further comprising, before the step of biologically pretreating the hemicellulose and lignin, removing cellulose from lignocellulosic biomass to fractionate hemicellulose and lignin.

6. The method of claim 1, wherein the red oleaginous yeast strain comprises one or more selected from the group consisting of Rhodosporidium toruloides, Rhodotorula glutinis, and Rhodotorula glutinis var. dairenensis.

7. The method of claim 1, wherein the red oleaginous yeast strain is comprised at a concentration of an optical density value of 0.01 to 16 at 600 nm in the medium.

8. The method of claim 1, wherein for the biological pretreatment product of the hemicellulose and lignin, monosaccharides, acetic acid, and ethanol in the medium are completely consumed by the red oleaginous yeast strain.

9. The method of claim 1, wherein the culturing in the biological pretreatment step is performed at 20° C. to 40° C. for 1 to 30 days.

10. The method of claim 1, wherein the culturing in the enzymatic saccharification step is performed at 40° C. to 60° C. for 0.5 to 10 days.
